(19) United States
(12) Patent Application Publication
(10) Pub. No.: US 2010/0274322 A1
(43) Pub. Date: Oct. 28, 2010

(54) TRACKING OF COMMUNICATION SESSIONS WITH AN IMPLANTABLE MEDICAL DEVICE

(76) Inventor: Touby A. Drew, Golden Valley, MN (US)

Correspondence Address: Medtronic - WK, P.O. Box 71355, Marietta, GA, 30007-1355, US

(21) Appl. No.: 12/428,019

(22) Filed: Apr. 22, 2009

(51) Int. Cl. A61N 1/08

(52) U.S. Cl. 607/60

(57) ABSTRACT

Communication sessions between external devices and implantable medical devices are tracked using a session value that is incremented for each new session and that is stored within the implantable medical device. Session logs that are created for each session may include the session value that is obtained for that session. The session value allows available session logs to be considered in the proper sequence and allows for an awareness of missing session logs. The incrementing of the session value may occur at the external device according to one convention, or at the implantable medical device according to another. The session value to be used for a given session may be the value accessed from the implantable medical device according to one convention or may be the session value that results from incrementing the session value that is accessed from the implantable medical device according to another.

### TECHNICAL FIELD

Embodiments are related to tracking of communication sessions. More particularly, embodiments are related to tracking of communication sessions between external devices and implantable medical devices.

### BACKGROUND

External devices and implantable medical devices (IMD) occasionally have a need to communicate. For instance, a clinician may need to review information stored within an IMD in order to assess a particular therapy and/or to modify therapy parameters. The IMD, such as a stimulator, drug delivery pump, and the like, may transfer the requested information to the external device, such as a device programmer, where it can be displayed, manipulated, and returned to the IMD.

It may be desirable to keep a session log that identifies the external device and the IMD that are involved in the session. The session log may also identify the date and time of the session, according to the clock of the particular programmer being used, as well as identifying the data obtained and any actions that took place during the session. The information being recorded to the session log may include information such as the therapy efficacy data retrieved from the IMD and the changes made by the clinician to the therapy parameters.

The session logs are typically stored in the external device and are later accessed by a clinician. The session logs allow the clinician to spot trends in efficacy of the therapy, correlate physiological response to given therapy parameters, and so forth. Viewing the session logs in the proper sequence may be necessary to properly assess the therapy, but the sequence may be difficult to determine.

Putting the session logs into the proper sequence requires all session logs to be available and to have reliable clock information. However, that may not always be achievable. For instance, different external devices may be used for different sessions for a single IMD. Those external devices may be located at different facilities, in different time zones, and so forth. As a result, one may not know whether session logs for the same IMD are also present on other external devices and/or whether, how many, or what, session logs are missing from an external device that created the session log. Session logs may be overlooked or poorly understood as a result. Furthermore, due to variations in time zones and clock settings, the clock information stored to the session log may not always be a reliable manner of putting available session logs into the proper order.

### SUMMARY

Embodiments address issues such as these and others by providing for tracking of communication sessions with a session value that is stored within the implantable medical device and that is updated for each communication session. The session value can be accessed by the external device and a session value can then be assigned to a given session log. Thus, regardless of how many external devices are used to establish a session with the IMD and regardless of how accurate associated clock information is, the session value present in the session log indicates the proper sequence of available session logs and provides awareness of missing session logs.

An embodiment provides a method of tracking communication sessions of an implantable medical device. The method involves establishing a communication session between the implantable medical device and an external device. During the communication session, a session value that is stored within a memory of the implantable medical device is obtained. During the communication session, the obtained session value is incremented to provide an incremented session value and the memory of the implantable medical device is updated with the incremented session value.

An embodiment provides an implantable medical device that includes a memory that stores medical therapy parameters and stores a session value. Communication circuitry exchanges signals carrying data with an external device during a communication session. Medical circuitry implements the therapy parameters to provide therapy. A processor provides data to the communication circuitry for delivery to the external device and receives data sent by the external device from the communication circuitry. The processor is responsive to an interrogation request by the external device for the session value stored in the memory by obtaining the session value from memory and providing the session value to the communication circuitry for delivery to the external device. The processor is further responsive to a write request by the external device that specifies an incremented session value by writing the incremented session value to the memory.

An embodiment provides an implantable medical device that also includes a memory that stores medical therapy parameters and stores a session value. Communication circuitry exchanges signals carrying data with an external device during a communication session. Medical circuitry implements the therapy parameters to provide therapy. A processor provides data to the communication circuitry for delivery to the external device and receives data sent by the external device from the communication circuitry. The processor is responsive to an interrogation request by the external device by obtaining the session value from memory, incrementing the session value to provide an incremented session value, and to write the incremented session value to the memory.

An embodiment provides an external device that communicates with an implantable medical device. Communication circuitry exchanges signals carrying data with the implantable medical device during a communication session. A processor provides data to the communication circuitry for delivery to the implantable medical device and receives data sent by the implantable medical device from the communication circuitry. The processor provides an interrogation request for a session value to the communication circuitry for delivery to the implantable medical device and receives the session value sent by the implantable medical device from the communication circuitry. The processor increments the received session value to provide an incremented session value and provides the incremented session value to the communication circuitry for delivery to the implantable medical device.

An embodiment provides an external device that communicates with an implantable medical device. Communication circuitry exchanges signals carrying data with the implantable medical device during a communication session. A processor provides data to the communication circuitry for delivery to the implantable medical device and receives data sent by the implantable medical device from the communication circuitry. The processor provides an interrogation request for a session value to the communication circuitry for delivery to the implantable medical device, receives the session value sent by the implantable medical device from the communication circuitry, and creates a session log including the received session value.

### DESCRIPTION OF THE DRAWINGS

FIG. 1 shows an operating environment for illustrative embodiments that track communication sessions by using a session value.

FIG. 2 shows an illustrative embodiment of an external device that communicates with an implantable medical device (IMD).

FIG. 3 shows an illustrative embodiment of an IMD that communicates with an external device.

FIG. 4 shows an example of a session log.

FIG. 5 shows an example of memory allocation within an IMD.

FIG. 6 shows examples of operational flow of an embodiment of an external device that increments a session value.

FIG. 7 shows examples of operational flow of an embodiment of an external device that communicates with an IMD that increments the session value.

FIG. 8 shows examples of operational flow of an embodiment of an IMD that communicates with an external device that increments the session value.

FIG. 9 shows examples of operational flow of an embodiment of an IMD that increments the session value.

FIG. 10 shows an example of an event report from an external device that indicates where sessions are missing.

FIG. 11 shows an example of a therapy report from an external device that indicates where sessions are missing.

### DETAILED DESCRIPTION

Communication sessions between external devices and implantable medical devices (IMD) are tracked using a session value that is stored within memory of the IMD. Each communication session results in the session value being incremented so that each communication session has a unique session value that establishes a sequence of the communication sessions. Session logs may be created with the session value added so that the session logs may be put into a proper sequence and so that there is an awareness of missing session logs.

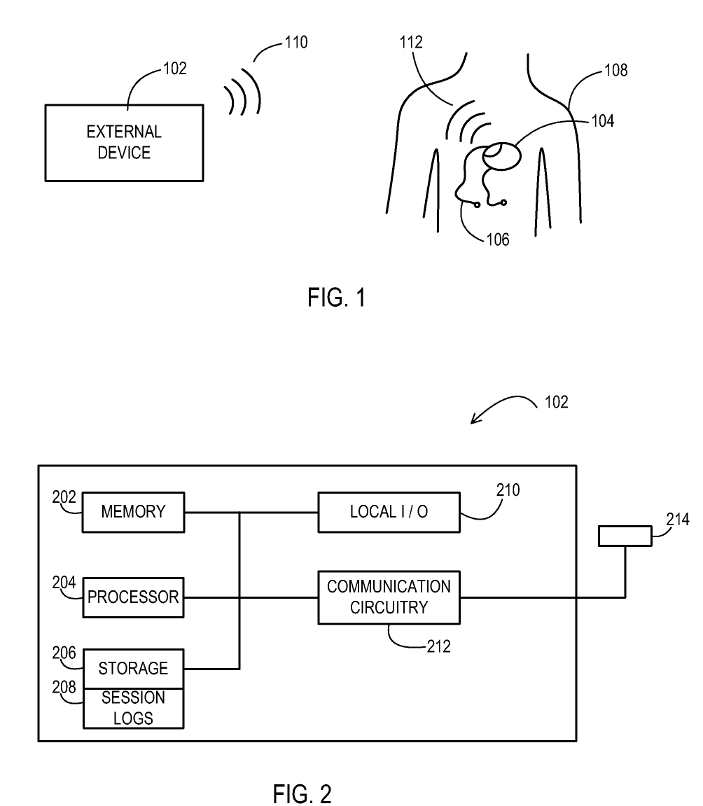

FIG. 1 shows an external device 102 in communication with an IMD 104 that is implanted within a patient 108. The external device 102 may be one of various device types, such as a clinician programmer, a patient programmer, a remote or distributed programmer system, or other IMDs. In some cases, the clinician or patient may communicate more directly with the IMD with a medical magnet, physical taps, brain interfaces, and the like. Likewise, the IMD 104 may be of various device types as well, such as a stimulator, a monitor, a drug delivery pump, a data collector, a sensing device, a trialing device that may be mounted externally to the patient and so forth. The IMD 104 may have medical components 106, such as stimulation, leads, monitoring leads, catheters, and the like.

The external device 102 and the IMD 104 typically communicate through a form of telemetry. In the case of a wireless communication link, wireless signals 110 are sent by the external device 102 and are received by the IMD 104. Likewise, wireless signals 112 are sent by the IMD 104 and are received by the external device 102. The wireless communication link may be a near field telemetry where the wireless signals 110 are generated by a wand in very close proximity to the IMD 104 and the wireless signals 112 are received by the wand. As another example, the telemetry may use radio frequency (RF) signaling where an antenna of the external device 102 and the IMD 104 are separated by a larger distance than occurs with near field telemetry to provide added convenience.

Typically, the external device 102 initiates a communication session with the IMD 104 by interrogating the IMD 104 for information. According to one or more of the embodiments, the external device 102 may also interrogate the IMD 104 to obtain a session value from the IMD 104. The session value can be used to keep track of sessions with the IMD 104. According to one embodiment, certain sessions may be exempt from session tracking due to the nature of the session. In such a case, the external device 102 may not interrogate the IMD 104 for the session value. Furthermore, for embodiments discussed below where the IMD 104 increments the session value, the IMD 104 may avoid incrementing the session value during an exempt session.

The external devices such as the external device 102 and the IMD 104 may utilize a standard convention in terms of whether the session value currently stored in the IMD 104 at the beginning of the session corresponds to the current session and is recorded for the current session, or whether this session value corresponds to the previous session and is to be incremented to provide a session value to be recorded for the current session. Furthermore, the external devices such as the external device 102 and the IMD 104 may utilize a standard convention in terms of whether the external device 102 or the IMD 104 increments the session value. Each device that may communicate with the IMD 104 follows the convention in use by the IMD 104 to maintain the integrity of the session value. These conventions are further discussed below.

FIG. 2 shows components of one example of the external device 102. The external device 102 includes a memory 202, a processor 204, and a storage device 206. The external device 102 also includes local input/output (I/O) ports 210 such as to provide local screen displays and to receive user input via keyboard, mouse, and so forth. The external device also includes communication circuitry 212 used to establish the telemetry to the IMD 104. The communication circuitry 212 may drive a signal propagation tool 214, such as a near field wand or an RF antenna.

The external device 102 may be one integrated device or may alternatively be a collection of devices linked together. For example, the external device 102 may be a combination of a programmer device coupled to a separate telemetry module via an external data connection.

The memory 202 may be used to store information in use by the processor 204. For instance, the memory 202 may temporarily store a session value that is to be incremented by the processor 204 or that is to be written to a session log. The memory may be of various types, such as volatile, non-volatile, or a combination.

The storage device 206 may be used to store information for a long term and may be of various types such as non-volatile so that the information is retained when the external device 102 is powered off. The session values may be eventually written to session logs 208 that are stored to the storage device 206. Examples of the storage device 206 include electronic, magnetic, and optical drives. The storage device 206 and the memory 202 are both examples of computer readable media that may store information in the form of computer programming, data structures, and the like.

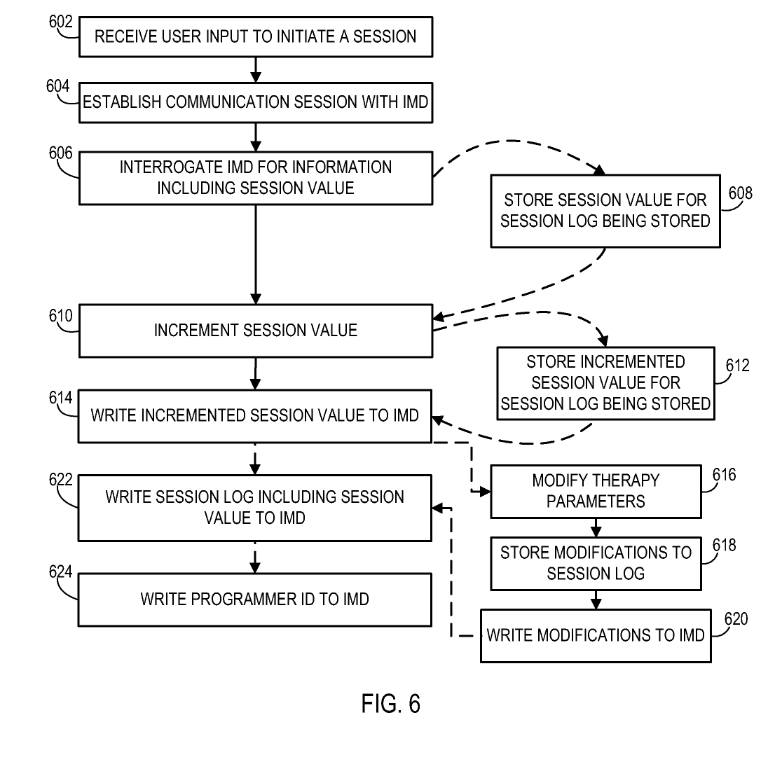

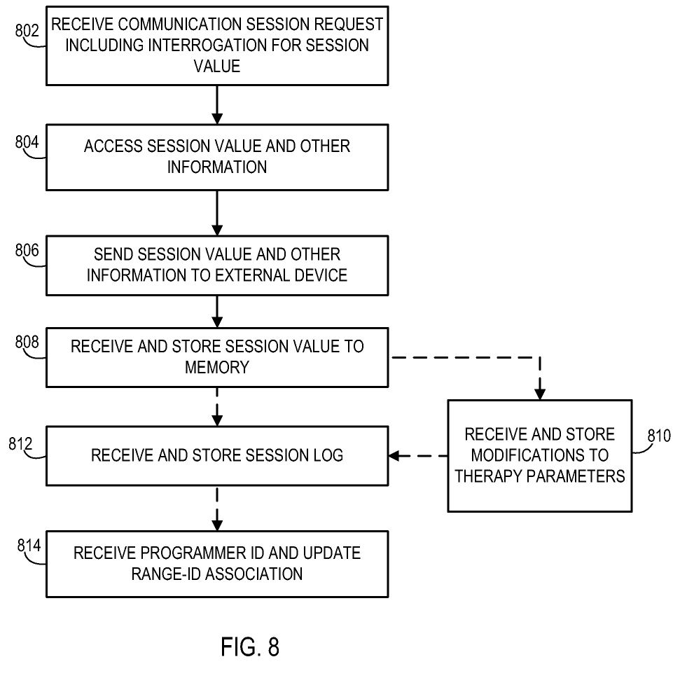

The processor 204 performs logical operations such as those of FIGS. 6 and 8 to allow communication sessions with the IMD 104 to occur and to track the communication sessions with a session value. The processor 204 may be of various forms. For instance, the processor 204 may be a general-purpose programmable processor that executes software that is stored on the storage device 208 or elsewhere. Other examples include a dedicated purpose hardware circuit or hard-wired digital logic. The processor 204 may communicate with the various other components through one or more data buses.

The external device 102 may implement applications that have session logic that is used to properly communicate with a given IMD 104 and to utilize a proper encoding and decoding to interpret information including the session value. As different models and types of IMDs exist, different applications may be provided for the different IMDs. To the extent a given application may communicate with different types of IMDs, the application may utilize a look-up table or deduction logic to determine the correct session logic. The session logic for a given IMD 104 may specify the memory location within the IMD 104 where the session value is stored so that the external device 102 may have more direct access to the session value without relying on additional logic in the IMD 104 to interpret a dedicated request. Alternatively, the IMD 104 may be configured to respond to a particular request for the value rather than exposing the memory location.

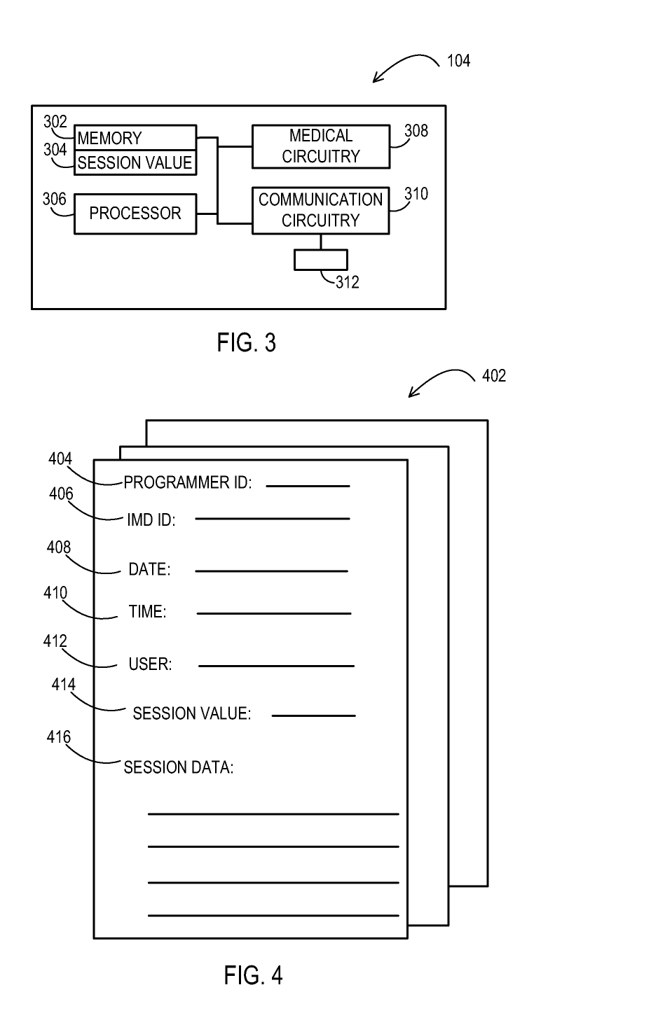

FIG. 3 shows components of one example of the IMD 104. The IMD 104 includes a memory 302 and a processor 306. The IMD 104 also includes medical circuitry 308 that performs a medical task such as stimulation, drug delivery, monitoring, and the like. The IMD 104 also includes communication circuitry 310 used to establish the telemetry to the external device 102. The communication circuitry 310 may drive a signal propagation tool 312, such as an integral near field or RF antenna.

The memory 302 may be used to store information in use by the processor 306. The memory 302 may also store a session value 304 that is accessed, incremented, and then overwritten back to the memory 302. The memory 302 may store additional information including therapy parameters that are used to control the medical circuitry 308. The memory 302 may also store session logs as well as store session value ranges in association with an identification of the external device 102 corresponding to the session value ranges. The memory 302 may be of various types such as volatile, non-volatile, or a combination of the two. The memory 302 is also an example of computer readable media that may store information in the form of computer programming, data structures, and the like.

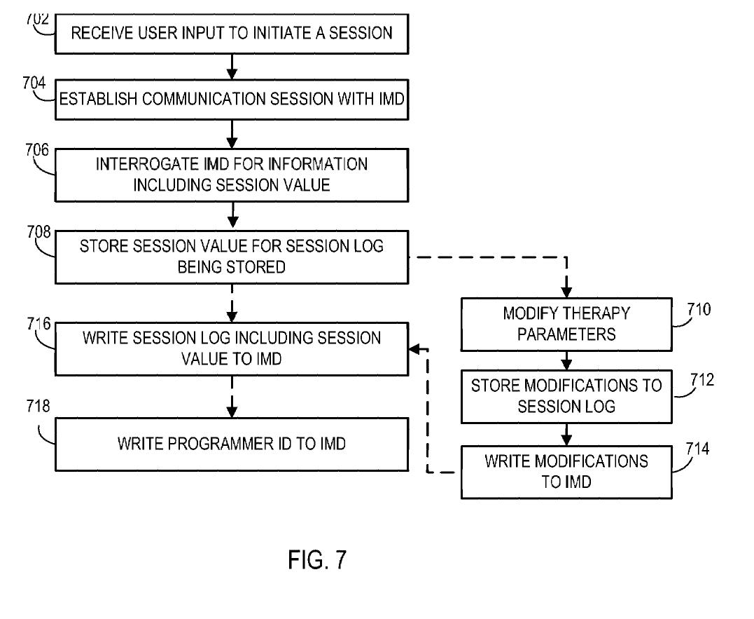

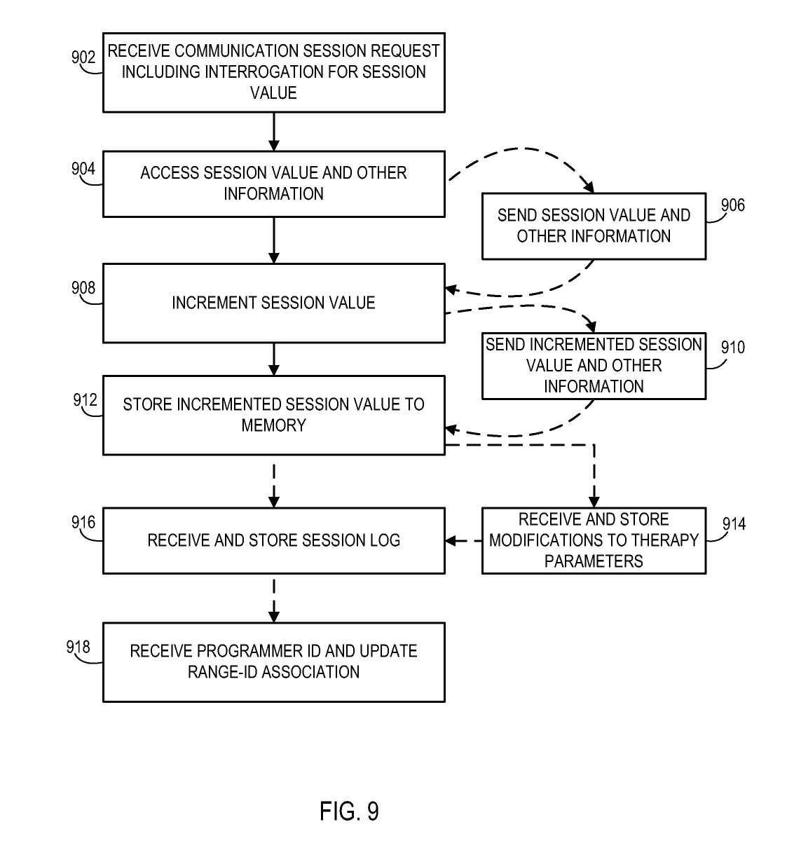

The processor 306 performs logical operations such as those of FIGS. 7 and 9 to allow communication sessions with the external device 102 to occur and to track the communication sessions with a session value. The processor 306 may be of various forms like those discussed above for the processor 204 of the external device 102. The processor 306 may communicate with the various other components through one or more data buses.

FIG. 4 shows an example of a session log 402 that may be generated by the external device 102 during a communication session with the IMD 104. The session log 402 may include several pieces of information. An identification 404 of the external device 102 may be present. Should the session log 402 be transferred to a different external device than the external device 102 that created the session log, the identification 404 may be useful to indicate where the session log originated. The session log 402 may also include an identification 406 of the IMD 104 that was involved in the communication session. This identification 406 ensures that the session log pertains to the particular IMD 104 of interest.

Other administrative information may also be included. A timestamp showing the date 408 and time 410 of the communication session may be present. An identification 412 of the entity (e.g. person or clinic) using the external device 102 to communicate with the IMD 104 may also be included.

Embodiments also provide for the session value 414 corresponding the communication session to be recorded to the session log 402. As discussed above, depending upon the convention in use between the external devices 102 and the IMD 104, the session value 414 may be the number read from the IMD 104 or may be the number that results from the external device 102 incrementing the session value that has been read from the IMD 104. The convention in use may call for the session value 414 to be a simple integer where the increment is by one. Alternatively, the convention in use may call for the session value 414 to be a more complex value and/or the increment to be other than by one.

The session log 402 may also include session data 416. The session data 416 may represent what information has been obtained from the IMD 104 during this session, any analysis performed on that data during this session, any modifications to operating parameters of the IMD 104 during this session, and so forth.

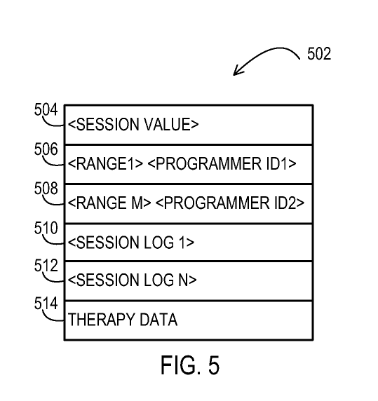

FIG. 5 shows an example of memory allocation 502 within the IMD 104. The memory 302 of the IMD 102 may include some or all of the information shown in this particular example of FIG. 5. In this example, a memory location 502 stores the session value. Here the session value may be accessed and a new session value that results from incrementing the existing one may be stored to overwrite the existing one.

Other information related to the communication sessions may be stored in the memory 302. For instance, a range of session values may be stored in association with an identification of the external device 102 that conducted those corresponding communication sessions at memory locations 506 through 508. In this example a first range of session values are stored in relation to a first external device identifier, while a second range of session values are stored in relation to a second external device identifier. Thus, if a clinician wishes to find and review a particular session log for a given IMD 104, where the session log is known but the location of the session log is not known, the clinician may have an external device interrogate the IMD 104 to obtain the external device identification that is associated with the range that includes the session value of the session log of interest. The clinician may then attempt to access the session log of interest from the identified external device or from the user, clinic, or medical record system that is associated with the session log or identified external device.

Session logs may also be stored in the memory 302 such as in the memory locations 510 through 512. This may aid the clinician in finding a session log of interest that is not available in the external device currently in use. To prevent the session logs from occupying an excessive amount of the memory 302, there may be a limited amount of memory 302 allocated for the session logs, and the session logs may be handled using a prioritization scheme. For instance, a first-in-first-out approach may be used so that the oldest session log is the one that is overwritten with the current session log when the allocated space is full. These session logs may include the session value that has been appended by the external device 102.

The memory 302 may also include therapy data at a memory location 514. The therapy data may include parameter settings, monitored values, and the like. The memory 302 may include other items not shown in FIG. 5 such as programming for the processor 306.

FIG. 6 shows the operational flow of one embodiment of an external device 102 for tracking the communication session by getting the session value from the IMD 104, incrementing the session value, and writing the incremented session value back to the IMD 104. The operational flow also provides alternate pathways to demonstrate both conventions regarding whether the stored session value from the IMD 104 applies to the current session or to the previous one. The alternative paths are illustrated as phantom lines.

The external device 102 receives a user input that requests a communication session be established with the IMD 104 at an input operation 602. The external device 102 then establishes the communication session with the IMD 104 at a communication operation 604. Once the telemetry link is established, the external device 102 interrogates the IMD 104 for information needed for the communication session, including the session value, at an interrogation operation 606.

At this point, the path of operational flow may differ depending upon the convention in use. For a convention that calls for the value already stored by the IMD 104 to be the session value for the present session, then the external device 102 stores the session value that has been received by the interrogation operation 606 at a storage operation 608. The session value may be queued in storage or memory for eventual inclusion in the session log being created for the current session. Thereafter, the external device 102 then increments the session value at an increment operation 610 for purposes of writing the incremented session value back to the IMD 104.

If the convention in use calls for the value already stored by the IMD 104 to be incremented so that the incremented session value is the session value for the current session, then operational flow proceeds directly from the interrogation operation 606 to the increment operation 610. After the session value is incremented to produce the incremented session value, the external device 102 stores the incremented session value at a storage operation 612. The incremented session value may be queued in storage or memory for eventual inclusion in the session log being created for the current session.

Either path then leads to a write operation 614 where the external device 104 writes the incremented session value back to the IMD 104. Prior to writing the incremented session value back to the IMD 104, the external device 102 may perform a check of the session value currently stored in the IMD 104. This check involves reading the session value from the IMD 104 again, and comparing it to the session value that was previously read from the IMD 104. This check ensures that no other intervening activities that might have resulted in a session value change have occurred, such as an intervening session with a different external device. If the session value is the same, then the write operation 614 occurs.

If such a change in the session value is found during the check, then the external device 102 may take various actions. In some cases such as for a non-critical IMD 104, the session value may be overwritten at the write operation 614. In other cases the external device 102 may take other actions such as outputting a notification of the issue so that a clinician can investigate the matter, clear out all device settings and prompt the clinician to re-interrogate the IMD 104 and re-configure any parameter changes, and so forth.

Additional operations may also be performed for various embodiments during the communication session with the IMD 104. For instance, the external device 102 may modify therapy parameters at a modification operation 616, may store such modifications to the session log at a storage operation 618, and may write such modifications to the IMD 104 at a write operation 620. Additionally or alternatively, the external device 102 may write the session log including the session value to the IMD 104 at a write operation 622. The external device 102 may also write an identification of the external device 102 to the IMD 104 for storage in association with a session value range that includes the current session value at a write operation 624.

FIG. 7 shows the operational flow of another embodiment of an external device 102 for tracking the communication session by getting the session value from the IMD 104 and storing the received session value to the session log for the current session. Some additional operations may also be included for various embodiments, and the additional paths to include those operations are illustrated as phantom lines.

The external device 102 receives a user input that requests a communication session be established with the IMD 104 at an input operation 702. The external device 102 then establishes the communication session with the IMD 104 at a communication operation 704. Once the telemetry link is established, the external device 102 interrogates the IMD 104 for information needed for the communication session, including the session value, at an interrogation operation 706.

The external device 102 stores the session value that has been received by the interrogation operation 706 at a storage operation 708. The session value may be queued in storage or memory for eventual inclusion in the session log being created for the current session. Because this convention relies on the IMD 104 to increment the session value, there is no increment operation and there is no operation to write an incremented session value to the IMD 104.

The additional operations may also be performed during the communication session with the IMD 104. For instance, the external device 102 may modify therapy parameters at a modification operation 710, may store such modifications to the session log at a storage operation 712, and may write such modifications to the IMD 104 at a write operation 714. Additionally or alternatively, the external device 102 may write the session log including the session value to the IMD 104 at a write operation 716. The external device 102 may also write an identification of the external device 102 to the IMD 104 for storage in association with a session value range that includes the current session value at a write operation 718.

FIG. 8 shows the operational flow of one embodiment of the IMD 104 for tracking the communication session by providing the session value to the external device 102, receiving the incremented session value from the external device 102, and storing the incremented session value in place of the previous session value. These operations are complementary to those of FIG. 6 for the external device 102. Some additional operations may also be included for various embodiments, and the additional paths to include those operations are illustrated as phantom lines.

The IMD 104 receives a communication session request from the external device 102 including an interrogation for the session value at an interrogation operation 802. The IMD 104 accesses the session value and any other requested information from the appropriate memory locations at an access operation 804. The IMD 104 then sends the session value and the other information to the external device 102 at a send operation 806. The IMD 104 then receives and stores to the memory the session value that has been incremented by the external device 102 at a storage operation 808.

The additional operations may also be performed during the communication session with the external device 102. For instance, where the external device 102 modifies therapy parameters, the IMD 104 may receive the modified therapy parameters and store them to memory at a storage operation 81 0. As another example, where the external device 102 writes the session log including the session value to the IMD 104, the IMD 104 then receives and stores the session log to memory at a storage operation 812. Where the external device 102 writes an identification of the external device 102 to the IMD 104, the IMD 104 then receives and stores the identification in association with the session range including the current session value a storage operation 814.

FIG. 9 shows the operational flow of another embodiment of the IMD 104 for tracking the communication session by providing the session value to the external device 102, incrementing the session value, and storing the incremented session value in place of the previous session value. These operations are complementary to those of FIG. 7 for the external device 102. The operational flow also provides alternate pathways to demonstrate both conventions regarding whether the session value being sent by the IMD 104 for application to the current session is the session value that is accessed from memory or an incremented session value. The alternative paths are illustrated as phantom lines.

The IMD 104 receives a communication session request from the external device 102 including an interrogation for the session value at an interrogation operation 902. The IMD 104 accesses the session value and any other requested information from the appropriate memory locations at an access operation 904.

At this point, the path of operational flow may differ depending upon the convention in use. For a convention that calls for the value already stored by the IMD 104 to be the session value for the present session, then the IMD 104 sends the session value that has been accessed from memory along with any other requested information to the external device 102 at a send operation 906. Thereafter, the IMD 104 then increments the session value at an increment operation 908 for purposes of storing the incremented session value in place of the previous session value in memory.

If the convention in use calls for the value already stored by the IMD 104 to be incremented so that the incremented session value is the session value for the current session, then operational flow proceeds directly from the access operation 904 to the increment operation 908. After the session value is incremented to produce the incremented session value, the IMD 104 sends the incremented session value along with any other requested information to the external device 102 at a send operation 910.

After having completed sending and incrementing the session value, both paths then proceed to a storage operation 912. Here, the IMD 104 stores the incremented session value to memory in place of the previous session value so that the incremented session value is available to be accessed during the next communication session.

The additional operations may also be performed during the communication session with the external device 102. For instance, where the external device 102 modifies therapy parameters, the IMD 104 may receive the modified therapy parameters and store them to memory at a storage operation 914. As another example, where the external device 102 writes the session log including the session value to the IMD 104, the IMD 104 then receives and stores the session log to memory at a storage operation 916. Where the external device 102 writes an identification of the external device 102 to the IMD 104, the IMD 104 then receives and stores the identification in association with the session range including the current session value a storage operation 918.

Tracking sessions with the session value as described above allows for additional analysis and support that may not be readily available otherwise. For instance, reports and/or graphics based on logged session information which use the session value to reconstruct trends and so on may include markings that indicate where and how many sessions are missing. Furthermore, support may be provided where a search may be focused on finding and transferring the missing files. For instance, a local programmer may submit a request to a central resource that collects and maintains session logs from any number of external devices, and the request may specify those session logs that the local programmer is missing in terms of the corresponding session values and identification of the IMD for those missing session logs.

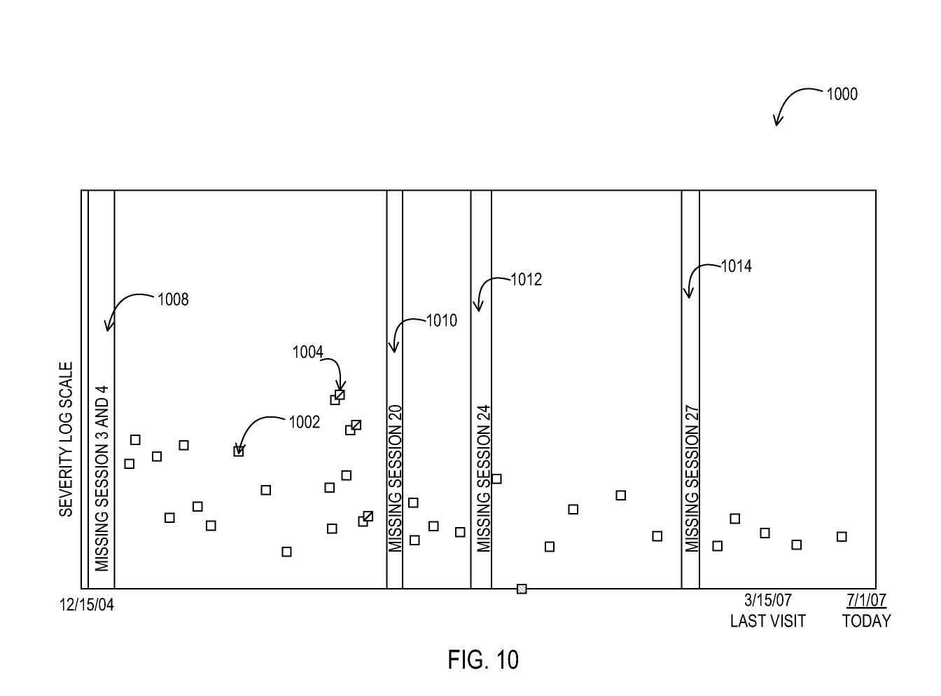

FIG. 10 shows a screenshot 1000 from the external device 102. The screenshot 1000 is an event report for a given patient that indicates when physiological events have taken place, such as neurological or cardiac events. Other events may also be included in such a report. For example, an event report may indicate device events such as a stalled motor or a reset; patient therapy events such as a bolus accepted/rejected, a device disabled, or medication administered; and/or clinician events such as changed programming for chronic therapy settings or acute therapy. The events are shown as dots 1002, with annotations added by clinicians for the events shown as dots 1004. In the example shown that pertains to physiological events, these event dots 1002 and annotation dots 1004 are distributed down a timeline 1006 and represent severity of the event that has occurred at the point in time. The event dots 1002 are present based on the external device 102 having successful sessions with the IMD 104 where the external device 102 retrieves the event data.

The report is built by accessing the session logs in order based on the session values. In this example, there are several instances where session values, and hence session logs, are missing. A first bar 1008 indicates that two consecutive sessions, namely sessions with session values 3 and 4, are missing while other bars 1010, 1012, and 1014 indicate that other session are also missing. Thus, a clinician can determine that the event data is incomplete and that some events may not be accounted for on the report of screenshot 1000. The clinician may then seek out those missing session logs based on the session values.

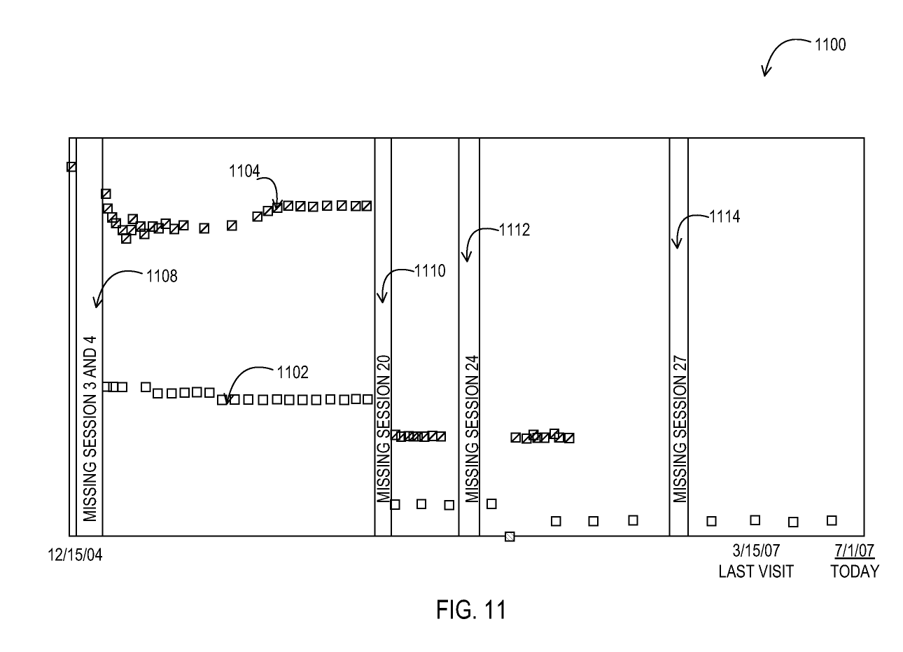

FIG. 11 shows a screenshot 1100 from the external device 102. The screenshot 1100 is a therapy report, such as specifying when a clinician or patient has requested a change to therapy and to what degree at given times and also specifying efficacy information related to the therapy at those times. The occurrences of change to therapy, such as changes to a total daily dosage of a drug, are shown as dots 1102, with efficacy ratings, such as a pain score, for the therapy shown as dots 1104. These therapy dots 1102 and efficacy dots 1104 are distributed down a timeline 1106. The therapy dots 1102 and efficacy dots 1104 are present based on the external device 102 having successful sessions with the IMD 104 where the external device 102 retrieves the therapy and efficacy data. It will be appreciated that the therapy dots 1102 may alternatively indicated when discrete applications of therapy occur rather than when changes to therapy occur. It will be further appreciated that the efficacy of the therapy could be automatically measured in a more continuous fashion, as opposed to discrete points in time, and could be shown in as a curve rather than in discrete efficacy dots 1104.

This report is also built by accessing the session logs in order based on the session values. In this example, the same instances where session values, and hence session logs, are missing are present. A first bar 1108 indicates that sessions with session values 3 and 4 are missing while other bars 1110, 1112, and 1114 indicate that other session are also missing. Thus, a clinician can determine that the therapy and efficacy data is incomplete and that some therapy occurrences may not be accounted for on the report of screenshot 1100. The clinician may then seek out those missing session logs based on the session values.

In particular, the clinician may note that prior to session 20, the dosage and the pain were both relatively severe. The clinician may further note that after session 20, the dosage and pain both dropped significantly. Therefore, the clinician may be curious to know what happened at missing session 20. Had the session value not been in place and used to indicate that session 20 is missing, the clinician might not have known of missing session 20 and might not have investigated the details of session 20.

While embodiments have been particularly shown and described, it will be understood by those skilled in the art that various other changes in the form and details may be made therein without departing from the spirit and scope of the invention.

What is claimed is:

1. A method of tracking communication sessions of an implantable medical device, comprising:
establishing a communication session between the implantable medical device and an external device;
during the communication session, obtaining a session value stored within a memory of the implantable medical device;
during the communication session, incrementing the obtained session value to provide an incremented session value; and
during the session, updating the memory of the implantable medical device with the incremented session value.

2. The method of claim 1, wherein obtaining the session value stored within the memory comprises interrogating the implantable medical device by the external device and receiving the session value at the external device.

3. The method of claim 2, wherein incrementing the obtained session value comprises incrementing the obtained session value by the external device.

4. The method of claim 2, wherein updating the memory comprises writing the incremented session value to the memory by the external device.

5. The method of claim 2, further comprising creating a session log at the external device that includes the obtained session number.

6. The method of claim 2, further comprising creating a session log at the external device that includes the incremented session number.

7. The method of claim 1, further comprising, during the session, obtaining therapy parameters from the memory of the implantable medical device at the external device, modifying the therapy parameters to provide modified therapy parameters, and writing the modified therapy parameters to the memory.

8. The method of claim 5, further comprising storing the session log at the external device.

9. The method of claim 5, further comprising writing the session log to the memory.

10. The method of claim 1, further comprising receiving an identification of the external device at the implantable medical device and storing the identification in association with the obtained session value separately from the incremented session value.

11. The method of claim 1, further comprising receiving an identification of the external device at the implantable medical device and storing the identification in association with the incremented session value separately from the incremented session value.

12. The method of claim 1, wherein obtaining the session value stored within the memory comprises accessing the session value by the implantable medical device.

13. The method of claim 12, wherein incrementing the obtained session value comprises incrementing the obtained session value by the implantable medical device.

14. The method of claim 12, wherein updating the memory comprises writing the incremented session value to the memory by the implantable medical device.

15. An implantable medical device, comprising:
a memory that stores medical therapy parameters and that stores a session value;
communication circuitry that exchanges signals carrying data with an external device during a communication session;
medical circuitry that implements the therapy parameters to provide therapy; and
a processor that provides data to the communication circuitry for delivery to the external device and that receives data sent by the external device from the communication circuitry, the processor being responsive to an interrogation request by the external device for the session value stored in the memory by obtaining the session value from memory and providing the session value to the communication circuitry for delivery to the external device, the processor being further responsive to a write request by the external device that specifies an incremented session value by writing the incremented session value to the memory.

16. The implantable medical device of claim 15, wherein the processor is further responsive to a second write request by the external device that specifies a session log by writing the session log to the memory.

17. The implantable medical device of claim 16, wherein the session log includes the session value.

18. The implantable medical device of claim 16, wherein the session log includes the incremented session value.

19. The implantable medical device of claim 15, wherein the processor is further responsive to a second write request by the external device that specifies an identification of the external device by writing the identification in association with the session value to the memory separately from the incremented session value.

20. The implantable medical device of claim 15, wherein the processor is further responsive to a second write request by the external device that specifies an identification of the external device by writing the identification in association with the incremented session value to the memory separately from the incremented session value.

21. An implantable medical device, comprising:
a memory that stores medical therapy parameters and that stores a session value;
communication circuitry that exchanges signals carrying data with an external device during a communication session;
medical circuitry that implements the therapy parameters to provide therapy; and
a processor that provides data to the communication circuitry for delivery to the external device and that receives data sent by the external device from the communication circuitry, the processor being responsive to an interrogation request by the external device by obtaining the session value from memory, incrementing the session value to provide an incremented session value, and to write the incremented session value to the memory.

22. The implantable medical device of claim 21, wherein the processor is further responsive to the interrogation request by the external device by providing the obtained session value to the communication circuitry for delivery to the external device.

23. The implantable medical device of claim 21, wherein the processor is further responsive to the interrogation request by the external device by providing the incremented session value to the communication circuitry for delivery to the external device.

24. The implantable medical device of claim 21, wherein the processor is responsive to a write request by the external device that specifies a session log by writing the session log to the memory.

25. The implantable medical device of claim 24, wherein the session log includes the session value.

26. The implantable medical device of claim 24, wherein the session log includes the incremented session value.

27. The implantable medical device of claim 21, wherein the processor responsive to a write request by the external device that specifies an identification of the external device by writing the identification in association with the session value to the memory separately from the incremented session value.

28. The implantable medical device of claim 21, wherein the processor is responsive to a write request by the external device that specifies an identification of the external device by writing the identification in association with the incremented session value to the memory separately from the incremented session value.

29. An external device that communicates with an implantable medical device, comprising:
communication circuitry that exchanges signals carrying data with the implantable medical device during a communication session; and
a processor that provides data to the communication circuitry for delivery to the implantable medical device and that receives data sent by the implantable medical device from the communication circuitry, the processor providing an interrogation request for a session value to the communication circuitry for delivery to the implantable medical device, receiving the session value sent by the implantable medical device from the communication circuitry, incrementing the received session value to provide an incremented session value, and providing the incremented session value to the communication circuitry for delivery to the implantable medical device.

30. The external device of claim 29, wherein the processor creates a session log including the received session value.

31. The external device of claim 30, further comprising a storage device, and wherein the processor stores the session log to the storage device.

32. The external device of claim 30, wherein the processor provides a write request specifying the session log to the communication circuitry for delivery to the implantable medical device.

33. The external device of claim 29, wherein the processor creates a session log including the incremented session value.

34. The external device of claim 29, wherein the processor provides an identification of the external device to the communication circuitry for delivery to the implantable medical device.

35. An external device that communicates with an implantable medical device, comprising:
communication circuitry that exchanges signals carrying data with the implantable medical device during a communication session; and
a processor that provides data to the communication circuitry for delivery to the implantable medical device and that receives data sent by the implantable medical device from the communication circuitry, the processor providing an interrogation request for a session value to the communication circuitry for delivery to the implantable medical device, receiving the session value sent by the implantable medical device from the communication circuitry, and creating a session log including the received session value.

36. The external device of claim 35, further comprising a storage device, and wherein the processor stores the session log to the storage device.

37. The external device of claim 35, wherein the processor provides a write request specifying the session log to the communication circuitry for delivery to the implantable medical device.

38. The external device of claim 35, wherein the processor provides an identification of the external device to the communication circuitry for delivery to the implantable medical device.

39. A system for tracking communication sessions, comprising:
an implantable medical device that stores medical therapy parameters and that stores a session value, and that implements the therapy parameters to provide therapy;
an external device that exchanges signals carrying data with the implantable medical device during a communication session, wherein the implantable medical device is responsive to an interrogation request by the external device for the session value by providing the session value to the external device, wherein the external device increments the session value, and wherein the implantable medical device is further responsive to a write request by the external device that specifies the incremented session value by storing the incremented session value.

40. A system for tracking communication sessions, comprising:
an implantable medical device that stores medical therapy parameters and that stores a session value, and that implements the therapy parameters to provide therapy;
an external device that exchanges signals carrying data with the implantable medical device during a communication session, wherein the implantable medical device is responsive to an interrogation request by the external device for the session value by incrementing the session value and storing the incremented session value.

41. A method of reporting information related to communication sessions between an external device and an implantable medical device, comprising:
displaying information obtained during a series of communication sessions represented by session logs;
detecting a missing communication session from the series based on reviewing the session logs containing unique session values and finding a missing session value; and
displaying an indicator in conjunction with the displayed information, the indicator specifying that information corresponding to the missing session is missing from the displayed information.

42. The method of claim 41, wherein displaying the indicator conveys which communication session is missing and when the communication session occurred relative to the communication sessions of the series whose information is being displayed.
